(19) United States
(12) Patent Application Publication
(10) Pub. No.: US 2025/0387015 A1
(43) Pub. Date: Dec. 25, 2025

(54) DRIVE DEVICE FOR MEDICAL MANIPULATOR, MEDICAL MANIPULATOR SYSTEM, AND MOTOR DEVICE FOR MEDICAL MANIPULATOR

(71) Applicant: OLYMPUS MEDICAL SYSTEMS CORP., Tokyo (JP)

(72) Inventor: Shu KAMBE, Hino-shi (JP)

(73) Assignee: OLYMPUS MEDICAL SYSTEMS CORP., Tokyo (JP)

(21) Appl. No.: 19/244,597

(22) Filed: Jun. 20, 2025

Related U.S. Application Data
(60) Provisional application No. 63/663,772, filed on Jun. 25, 2024.

(51) Int. Cl. A61B 1/00

(52) U.S. Cl. CPC A61B 1/0016; A61B 1/00057; A61B 1/00128

(57) ABSTRACT

A drive device for a medical manipulator includes: a housing; a motor; a support member that supports the motor, a flexure body having a first connector fixed to the housing, a second connector detachably connected to the support member, and a flexure portion provided between the first connector and the second connector, a strain sensor disposed in the flexure portion, wherein the second connector is fixed relative to the support member in a circumferential direction of the motor when the support member is attached.

### CROSS-REFERENCE TO RELATED APPLICATION

Priority is claimed on U.S. Provisional Application No. 63/663,772, filed on Jun. 25, 2024, the content of which is incorporated herein by reference.

### FIELD

The present disclosure relates to a drive device for a medical manipulator, a medical manipulator system, and a motor device for a medical manipulator.

### BACKGROUND

Conventionally, a medical manipulator system has been used for observing and treating the inside of a hollow organ such as the digestive tract. In the medical manipulator system, an insertion portion, a bending portion, and the like which are inserted into a hollow organ can be driven to bend electrically. A user can control the bending operation of the insertion portion and the like from an operation portion disposed outside a body.

A wire and the like are attached to the insertion portion and the like. When a motor drives the wire and the like, the insertion portion and the like are bent. A drive device (motor assembly) equipped with a motor can control the motor that drives the wire and the like by sensing the torque of the motor that drives the wire and the like. Japanese Unexamined Patent Application, First Publication No. 2019-144236 (which is hereinafter referred to as Patent Document 1) describes a torque transducer that can be attached to a motor and is capable of sensing torque.

The motor in the drive device of the medical manipulator needs to accurately bend the insertion portion that is inserted into the body and the like in order to properly perform observation and treatment using the medical manipulator.

Therefore, it is desirable for the drive device of the medical manipulator to be able to easily replace the motor with a new motor according to the deterioration state of the motor and the like.

Although the conventional drive device described in Patent Document 1 and the like can sense torque, it is not necessarily a structure in which the motor can be easily replaced.

### SUMMARY

The present disclosure provides a drive device for a medical manipulator, a medical manipulator system, and a motor device for a medical manipulator that are capable of sensing torque of a motor for driving a medical manipulator and that allow easy replacement of the motor.

A drive device for a medical manipulator according to a first aspect of the present disclosure includes: a housing; a motor; a support member that supports the motor; a flexure body having a first connector fixed to the housing, a second connector detachably connected to the support member, and a flexure portion provided between the first connector and the second connector, and a strain sensor disposed in the flexure portion, wherein the second connector is fixed relative to the support member in a circumferential direction of the motor when the support member is attached.

According to the drive device for the medical manipulator, the medical manipulator system, and the motor device for the medical manipulator of the present disclosure, it is possible to sense the torque of the motor for driving the medical manipulator and to allow easy replacement of the motor.

### BRIEF DESCRIPTION OF THE DRAWINGS

FIG. 1 is an overall view of a medical manipulator system according to a first embodiment.

FIG. 2 is a diagram illustrating an endoscope and an operation device of the medical manipulator system used by a surgeon.

FIG. 3 is a diagram illustrating an insertion portion of the endoscope.

FIG. 4 is a cross-sectional view illustrating a part of a bending portion of the insertion portion.

FIG. 5 is an enlarged view of a joint ring of the bending portion in a region E illustrated in FIG. 4.

FIG. 6 is a cross-sectional view of the bending portion taken along a line C1-C1 in FIGS. 4 and 5.

FIG. 7 is a view illustrating a first detachable portion before being attached to a drive device for the medical manipulator system.

FIG. 8 is a diagram illustrating a vertical bending wire attaching/detaching portion before being attached to the drive device.

FIG. 9 is a diagram illustrating the vertical bending wire attaching/detaching portion attached to the drive device.

FIG. 10 is a functional block diagram of the drive device.

FIG. 11 is a diagram illustrating a first motor unit and a first torque sensor.

FIG. 12 is a cross-sectional view of the first motor unit and the first torque sensor.

FIG. 13 is a cross-sectional view of the first motor unit detached from a fixed member.

FIG. 14 is a diagram illustrating a flexure body of the first torque sensor.

FIG. 15 is a perspective view of an operation device of the medical manipulator system.

FIG. 16 is a functional block diagram of a video control device of the medical manipulator system.

FIG. 17 is a control flowchart of a drive controller of a control device of the medical manipulator system.

FIG. 18 is a diagram illustrating a modified example of the flexure body.

FIG. 19 is a diagram illustrating another modified example of the flexure body.

FIG. 20 is a diagram illustrating still another modified example of the flexure body.

FIG. 21 is a diagram illustrating still another modified example of the flexure body.

FIG. 22 is a diagram illustrating the first motor unit and the first torque sensor of the medical manipulator system according to the first embodiment.

FIG. 23 is a cross-sectional view of the first motor unit and the first torque sensor.

FIG. 24 is a cross-sectional view of the first motor unit detached from the fixed member.

FIG. 25 is a perspective view of the flexure body.

### DETAILED DESCRIPTION

### First Embodiment

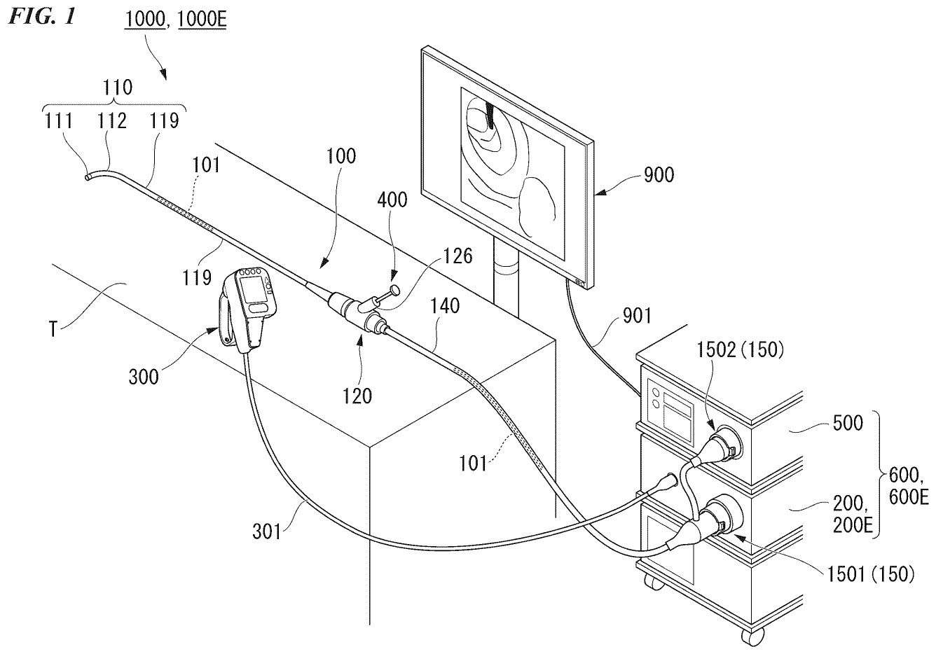

An electric endoscope system 1000 according to a first embodiment of the present disclosure will be described with reference to FIGS. 1 to 17. FIG. 1 is an overall view of the electric endoscope system 1000 according to this embodiment. The electric endoscope system 1000 is an example of a medical manipulator system. Examples of the medical manipulator include electrically driven endoscopes, catheters, treatment tools, endoluminal devices, and the like that are inserted into the body.

### [Electric Endoscope System 1000]

As illustrated in FIG. 1, the electric endoscope system 1000 is a medical system for observing and treating the inside of the body of a patient P lying on an operating table T. The electric endoscope system 1000 includes an endoscope 100, a drive device 200, an operation device 300, a treatment tool 400, a video control device 500, and a display device 900.

The endoscope 100 is a device that is inserted into the lumen of the patient P to observe and treat the affected area. The endoscope 100 is attachable to and detachable from the drive device 200. An internal passage 101 is formed inside the endoscope 100. In the following description, in the endoscope 100, the side that is inserted into the lumen of the patient P is referred to as the “distal end side A1,” and the side that is attached to the drive device 200 is referred to as the “proximal end side A2.”

The drive device 200 is detachably connected to the endoscope 100 and the operation device 300. The drive device 200 drives a built-in motor based on an operation input to the operation device 300 to electrically drive the endoscope 100. Further, the drive device 200 drives a built-in pump and the like based on an operation input to the operation device 300 to cause the endoscope 100 to perform air supply and suction.

The operation device 300 is detachably connected to the drive device 200 via an operation cable 301. The operation device 300 may be capable of communicating with the drive device 200 by wireless communication instead of wired communication. A surgeon S can electrically drive the endoscope 100 by operating the operation device 300.

The treatment tool 400 is a device that is inserted through the internal passage 101 of the endoscope 100 into the lumen of the patient P to treat an affected area. In FIG. 1, the treatment tool 400 is inserted into an internal path 101 of the endoscope 100 from a forceps port 126.

The video control device 500 is detachably connected to the endoscope 100 and acquires captured images from the endoscope 100. The video control device 500 causes the display device 900 to display captured images acquired from the endoscope 100, as well as GUI images and CG images intended to provide information to the operator.

The drive device 200 and the video control device 500 constitute a control device 600 that controls the electric endoscope system 1000. The control device 600 may further include a peripheral device such as a video printer. The drive device 200 and the video control device 500 may be integrated into one device.

The display device 900 is a device capable of displaying images such as an LCD. The display device 900 is connected to the video control device 500 via a display cable 901.

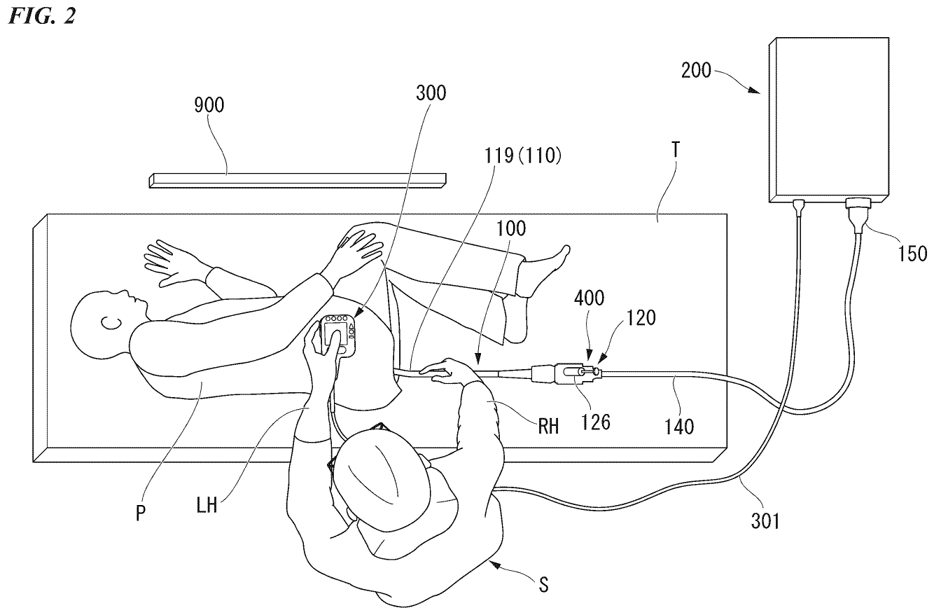

FIG. 2 is a diagram illustrating the endoscope 100 and the operation device 300 used by the surgeon S.

For example, the surgeon S operates the endoscope 100 inserted into the cavity from the anus of the patient P with his/her right hand RH and operates the operation device 300 with his/her left hand LH while observing the captured image displayed on the display device 900. Since the endoscope 100 and the operation device 300 are separated from each other, the surgeon S can operate the endoscope 100 and the operation device 300 independently without affecting each other.

### [Endoscope 100]

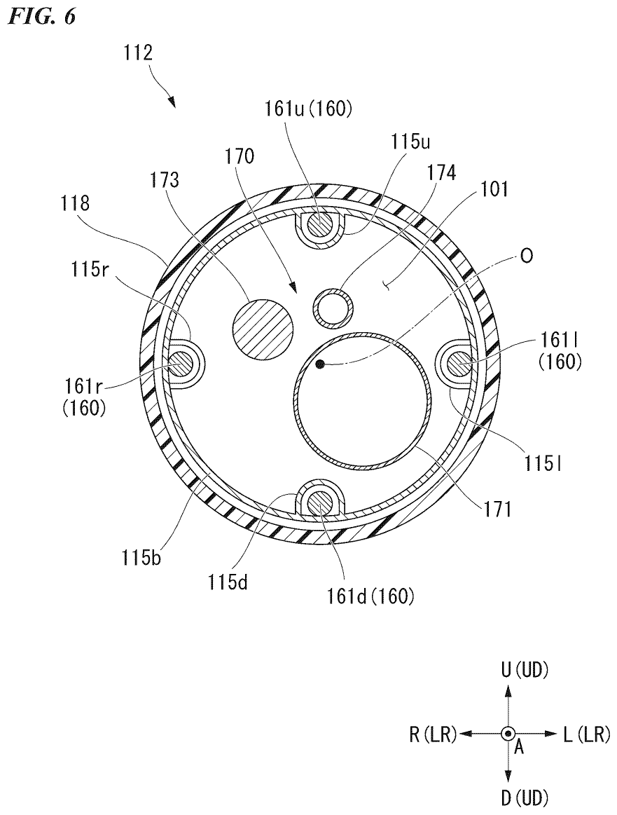

As illustrated in FIG. 1, the endoscope 100 includes an insertion portion 110, a connection portion (connector) 120, an external flexible portion 140, a detachable portion 150, a bending wire 160 (see FIG. 6), and an internal structure 170 (see FIG. 6). The insertion portion 110, the connection portion 120, the external flexible portion 140, and the detachable portion 150 are connected in this order from the distal end side.

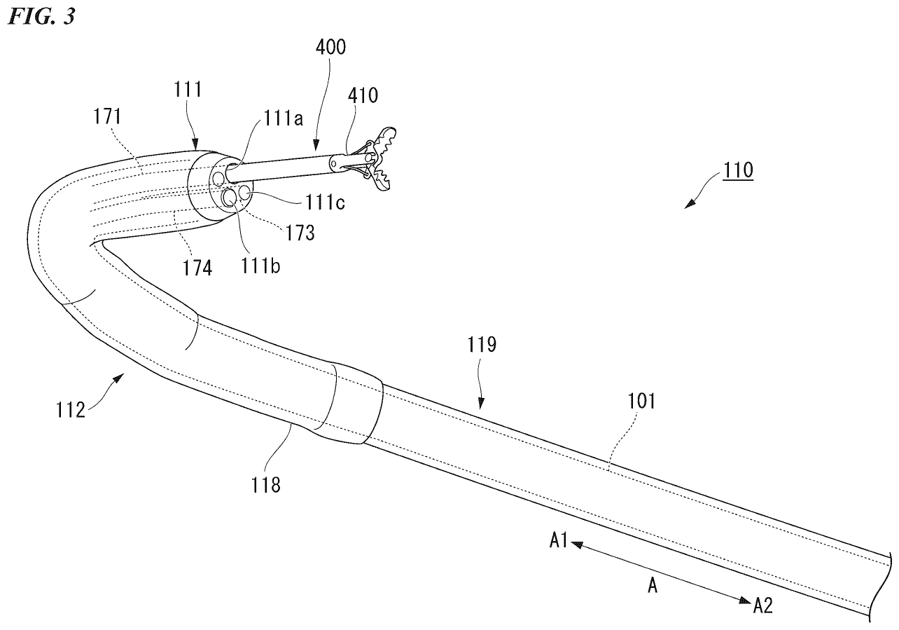

FIG. 3 is a diagram illustrating the insertion portion 110 of the endoscope 100.

The internal passage 101 is formed inside the endoscope 100 and extends from the distal end of the insertion portion 110 to the proximal end of the detachable portion 150 along the longitudinal direction A of the endoscope 100. The bending wire 160 and the internal structure 170 are inserted into the internal passage 101.

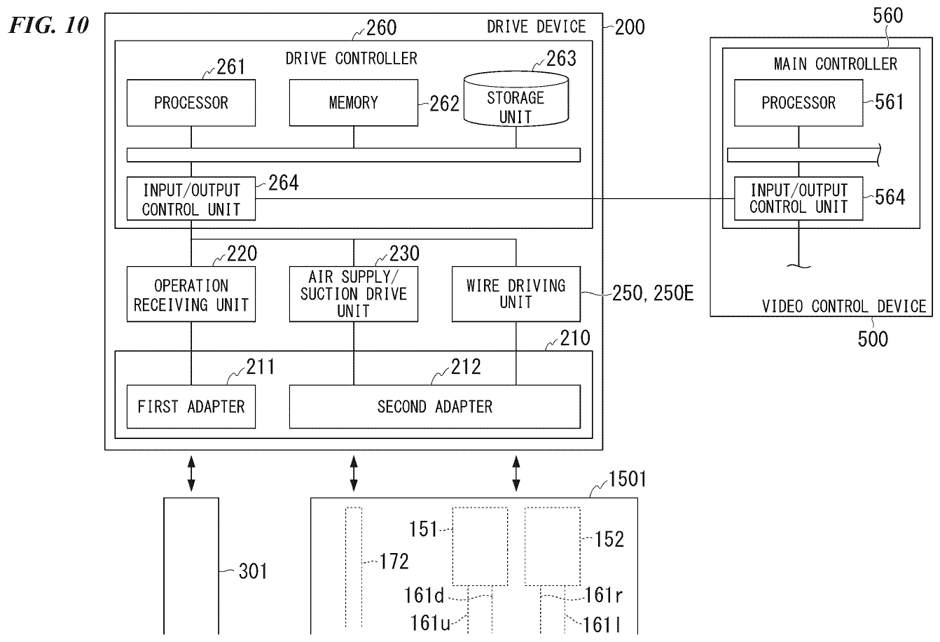

The internal structure 170 includes a channel tube 171, an air supply/suction tube 172 (see FIG. 10), an imaging cable 173, and a light guide 174.

### [Insertion Portion 110]

The insertion portion 110 is a long and thin member that can be inserted into a lumen. The insertion portion 110 has a distal end portion 111, a bending portion 112, and an internal flexible portion 119. The distal end portion 111, the bending portion 112, and the internal flexible portion 119 are connected in this order from the distal end side.

As illustrated in FIG. 3, the distal end portion 111 has an opening portion 111a, an illumination unit 111b, and an imaging unit 111c. The opening portion 111a is an opening that communicates with the channel tube 171. As illustrated in FIG. 3, a treatment portion 410 such as a grasping forceps provided at the distal end of the treatment tool 400 that passes through the channel tube 171 protrudes and retracts from the opening portion 111a.

The illumination unit 111b is connected to a light guide 174 that guides the illumination light, and emits the illumination light that illuminates the imaging target. The imaging unit 111c includes an imaging element such as a CMOS, and captures an image of the imaging target. An imaging signal is sent to the video control device 500 via the imaging cable 173.

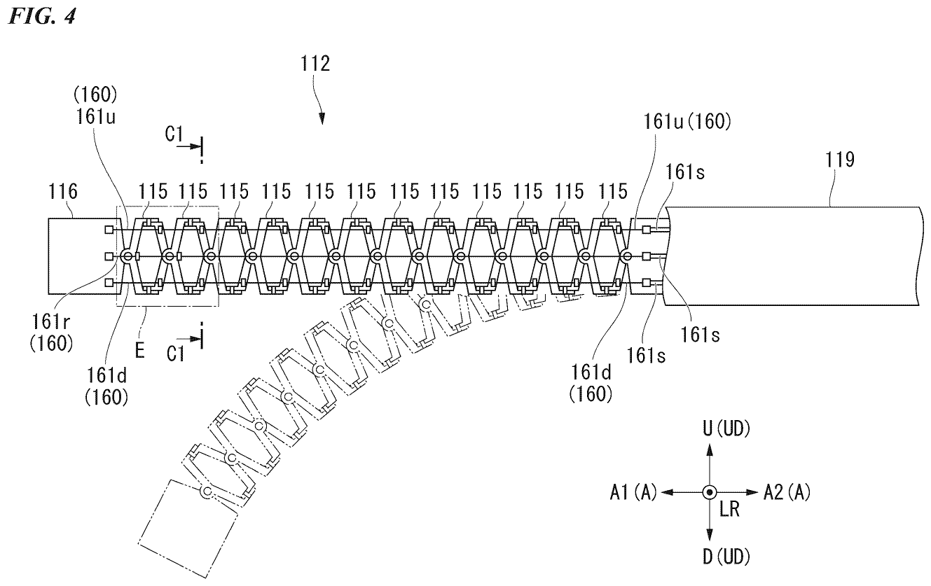

FIG. 4 is a cross-sectional view illustrating a part of the bending portion 112.

The bending portion 112 has a plurality of joint rings (also called bending pieces) 115, a distal end portion 116 connected to the distal ends of the plurality of joint rings 115, and an outer sheath 118 (see FIG. 3). The plurality of joint rings 115 and the distal end portion 116 are connected in the longitudinal direction A inside the outer sheath 118. Furthermore, the shape and number of the joint rings 115 of the bending portion 112 are not limited to those illustrated in FIG. 4. The bending portion 112 is an example of the movable portion of the medical manipulator.

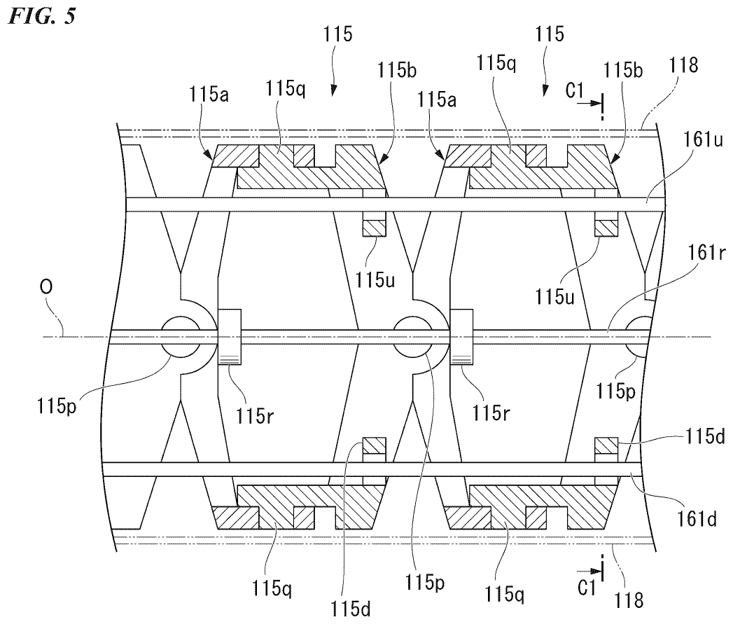

FIG. 5 is an enlarged view of the joint ring 115 in a region E illustrated in FIG. 4.

The joint ring 115 is a short cylindrical member made of metal. The plurality of joint rings 115 are connected to each other so that the internal spaces of the adjacent joint rings 115 are continuous.

The joint ring 115 has a first joint ring 115a on the distal end side and a second joint ring 115b on the proximal end side. The first joint ring 115a and the second joint ring 115b are connected to be rotatable in the up and down direction perpendicular to the longitudinal direction A (also referred to as the “UD direction”) by a first rotation pin 115p.

In the adjacent joint rings 115, the second joint ring 115b in the distal end side joint ring 115 and the first joint ring 115a in the proximal end side joint ring 115 are connected by a second rotation pin 115q to be rotatable in a left and right direction (also referred to as the “LR direction”) perpendicular to the longitudinal direction A and the UD direction.

The first joint ring 115a and the second joint ring 115b are alternately connected by the first rotation pin 115p and the second rotation pin 115q, and the bending portion 112 is freely bendable in a desired direction.

FIG. 6 is a cross-sectional view of the bending portion 112 taken along a line C1-C1 of FIGS. 4 and 5.

An upper wire guide 115u and a lower wire guide 115d are formed on the inner peripheral surface of the second joint ring 115b. The upper wire guide 115u and the lower wire guide 115d are arranged on both sides of the UD direction with the center axis O of the longitudinal direction A interposed therebetween. A left wire guide 1151 and a right wire guide 115r are formed on the inner peripheral surface of the first joint ring 115a. The left wire guide 1151 and the right wire guide 115r are arranged on both sides of the LR direction with the center axis O of the longitudinal direction A interposed therebetween.

The upper wire guide 115u, the lower wire guide 115d, the left wire guide 1151, and the right wire guide 115r have through holes formed along the longitudinal direction A, through which the bending wire 160 passes.

The bending wire 160 is a wire that bends the bending portion 112. The bending wire 160 extends to the detachable portion 150 through the internal passage 101. As illustrated in FIGS. 4 and 6, the bending wire 160 has an upper bending wire 161u, a lower bending wire 161d, a left bending wire 1611, a right bending wire 161r, and four wire sheaths 161s.

As illustrated in FIG. 4, each of the upper bending wire 161u, the lower bending wire 161d, the left bending wire 1611, and the right bending wire 161r is inserted through the wire sheath 161s. The distal end of the wire sheath 161s is attached to the joint ring 115 at the proximal end of the bending portion 112. The wire sheath 161s extends to the detachable portion 150.

The upper bending wire 161u and the lower bending wire 161d are wires that bend the bending portion 112 in the UD direction. The upper bending wire 161u is inserted through the upper wire guide 115u. The lower bending wire 161d is inserted through the lower wire guide 115d.

As illustrated in FIG. 4, the distal ends of the upper bending wire 161u and the lower bending wire 161d are fixed to the distal end portion 116 at the distal end of the bending portion 112. The distal ends of the upper bending wire 161u and the lower bending wire 161d fixed to the distal end portion 116 are arranged on both sides of the UD direction with the center axis O of the longitudinal direction A interposed therebetween.

The left bending wire 1611 and the right bending wire 161r are wires that bend the bending portion 112 in the LR direction. The left bending wire 1611 is inserted through the left wire guide 1151. The right bending wire 161r is inserted through the right wire guide 115r.

As illustrated in FIG. 4, the distal ends of the left bending wire 1611 and the right bending wire 161r are fixed to the distal end portion 116 of the bending portion 112. The distal ends of the left bending wire 1611 and the right bending wire 161r fixed to the distal end portion 116 are arranged on both sides of the LR direction with the center axis O of the longitudinal direction A interposed therebetween.

The bending portion 112 is freely bendable in a desired direction by pulling or relaxing each bending wire 160 (the upper bending wire 161u, the lower bending wire 161d, the left bending wire 1611, and the right bending wire 161r).

As illustrated in FIG. 6, the bending wire 160, the channel tube 171, the imaging cable 173, and the light guide 174 are inserted through the internal passage 101 formed inside the bending portion 112.

The internal flexible portion 119 is a long and flexible tubular member. The bending wire 160, the channel tube 171, the imaging cable 173, and the light guide 174 are inserted through the internal passage 101 formed in the internal flexible portion 119.

### [Connection Portion 120]

As illustrated in FIG. 1, the connection portion 120 is a member that connects the internal flexible portion 119 of the insertion portion 110 and the external flexible portion 140. The connection portion 120 has the forceps port 126 which is an insertion port for inserting the treatment tool 400.

### [External Flexible Portion 140]

The external flexible portion 140 is a long tubular member. The bending wire 160, the imaging cable 173, the light guide 174, and the air supply/suction tube 172 (see FIG. 10) are inserted through the internal passage 101 formed inside the external flexible portion 140.

### [Detachable Portion 150]

As illustrated in FIG. 1, the detachable portion 150 has a first detachable portion 1501 which is attached to the drive device 200 and a second detachable portion 1502 which is attached to the video control device 500. Furthermore, the first detachable portion 1501 and the second detachable portion 1502 may be an integrated detachable portion.

The internal passage 101 formed inside the external flexible portion 140 branches into the first detachable portion 1501 and the second detachable portion 1502. The bending wire 160 and the air supply/suction tube 172 are inserted through the first detachable portion 1501. The imaging cable 173 and the light guide 174 are inserted through the second detachable portion 1502.

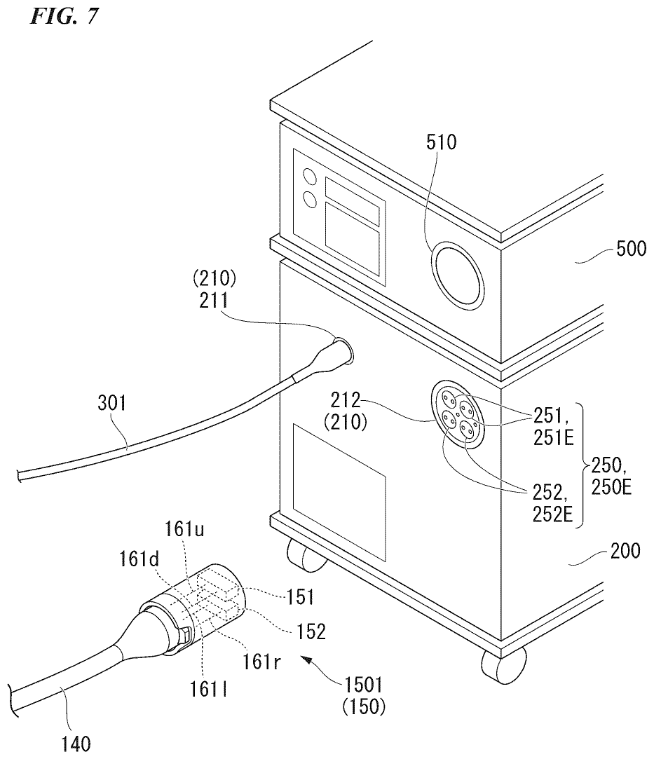

FIG. 7 is a diagram illustrating the first detachable portion 1501 before being attached to the drive device 200.

The first detachable portion 1501 has a vertical bending wire attaching/detaching portion 151 and a horizontal bending wire attaching/detaching portion 152.

The vertical bending wire attaching/detaching portion 151 is a mechanism that detachably connects the wires (the upper bending wire 161u and the lower bending wire 161d) that bend the bending portion 112 in the UD direction to the drive device 200.

The horizontal bending wire attaching/detaching portion 152 is a mechanism that detachably connects the wires (the left bending wire 1611 and the right bending wire 161r) that bend the bending portion 112 in the LR direction to the drive device 200.

The horizontal bending wire attaching/detaching portion 152 has the same structure as the vertical bending wire attaching/detaching portion 151, and therefore will not be illustrated or described.

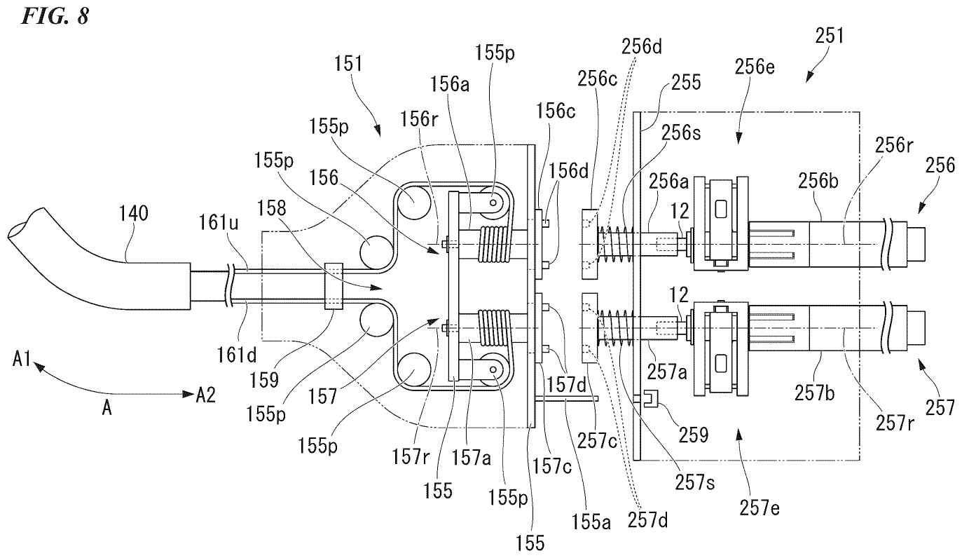

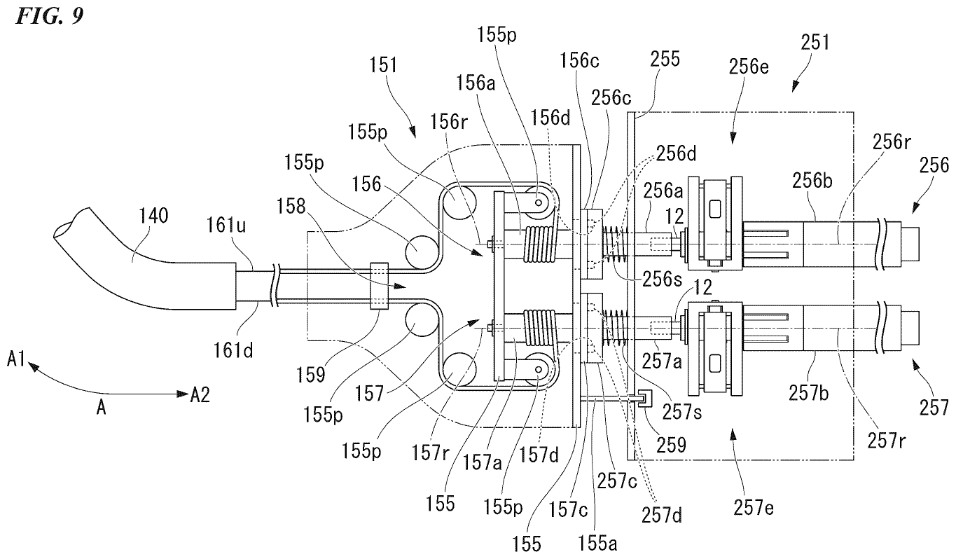

FIG. 8 is a diagram illustrating the vertical bending wire attaching/detaching portion 151 before being attached to the drive device 200. FIG. 9 is a diagram illustrating the vertical bending wire attaching/detaching portion 151 attached to the drive device 200. The vertical bending wire attaching/detaching portion 151 has a support member 155, a first rotation drum 156, a second rotation drum 157, and a tension sensor 159.

The support member 155 supports the first rotation drum 156, the second rotation drum 157, and the connection member 158. The support member 155 has an attachment/detachment detection dog 155a which is exposed on the proximal end side of the vertical bending wire attaching/detaching portion 151, and a plurality of bend pulleys 155p.

The bend pulley 155p changes the conveying direction of the upper bending wire 161u inserted through the external flexible portion 140 and guides the upper bending wire 161u to the first rotation drum 156. Further, the bend pulley 155p changes the conveying direction of the lower bending wire 161d inserted through the external flexible portion 140 and guides the lower bending wire 161d to the second rotation drum 157.

The first rotation drum 156 is supported by the support member 155 to be rotatable around a first drum rotation axis 156r extending along the longitudinal direction A. The first rotation drum 156 has a first winding pulley 156a and a first coupling portion 156c.

The first winding pulley 156a rotates around the first drum rotation axis 156r to pull or send the upper bending wire 161u. When the first winding pulley 156a rotates clockwise as viewed from the distal end side to the proximal end side, the upper bending wire 161u is wound around the first winding pulley 156a and pulled. Conversely, when the first winding pulley 156a rotates counterclockwise, the upper bending wire 161u is delivered from the first winding pulley 156a. With this configuration, even if the upper bending wire 161u retracts and advances by a large amount, the pulled portion is stored compactly and does not take up space.

The first coupling portion 156c is a disk member that rotates around the first drum rotation axis 156r. The first coupling portion 156c is fixed to the proximal end of the first winding pulley 156a and rotates integrally with the first winding pulley 156a. The first coupling portion 156c is exposed to the proximal end side of the vertical bending wire attaching/detaching portion 151. Two first fitting convex portions 156d are formed on the proximal end side surface of the first coupling portion 156c. Two first fitting convex portions 156d are formed on both sides with the first drum rotation axis 156r interposed therebetween.

The second rotation drum 157 is supported by the support member 155 to be rotatable around a second drum rotation axis 157r extending along the longitudinal direction A. The second rotation drum 157 has a second winding pulley 157a and a second coupling portion 157c.

The second winding pulley 157a rotates around the second drum rotation axis 157r to pull or send the lower bending wire 161d. When the second winding pulley 157a rotates counterclockwise as viewed from the distal end side to the proximal end side, the lower bending wire 161d is wound around the second winding pulley 157a and pulled. Conversely, when the second winding pulley 157a rotates clockwise, the lower bending wire 161d is delivered from the second winding pulley 157a.

The second coupling portion 157c is a disk member that rotates around the second drum rotation axis 157r. The second coupling portion 157c is fixed to the proximal end of the second winding pulley 157a and rotates integrally with the second winding pulley 157a. The second coupling portion 157c is exposed to the proximal end side of the vertical bending wire attaching/detaching portion 151. Two second fitting convex portions 157d are formed on the proximal end side surface of the second coupling portion 157c. Two second fitting convex portions 157d are formed on both sides with the second drum rotation axis 157r interposed therebetween.

The tension sensor 159 detects the tension of the upper bending wire 161u and the lower bending wire 161d. The detection result of the tension sensor 159 is acquired by a drive controller 260.

### [Drive Device 200]

FIG. 10 is a functional block diagram of the drive device 200.

The drive device 200 includes an adapter 210, an operation receiving unit 220, an air supply/suction drive unit 230, a wire driving unit (actuator) 250, and a drive controller 260.

As illustrated in FIG. 7, the adapter 210 includes a first adapter 211 and a second adapter 212. The first adapter 211 is an adapter to which the operation cable 301 is detachably connected. The second adapter 212 is an adapter to which the first detachable portion 1501 of the endoscope 100 is detachably connected.

The operation receiving unit 220 receives an operation input from the operation device 300 via the operation cable 301. When the operation device 300 and the drive device 200 communicate with each other by wireless communication rather than by wired communication, the operation receiving unit 220 has a known wireless receiving module.

The air supply/suction drive unit 230 is connected to the air supply/suction tube 172 inserted into the internal passage 101 of the endoscope 100. The air supply/suction drive unit 230 includes a pump or the like and supplies air to the air supply/suction tube 172. Further, the air supply/suction drive unit 230 sucks air from the air supply/suction tube 172.

The wire driving unit (actuator) 250 is coupled with the vertical bending wire attaching/detaching portion 151 and the horizontal bending wire attaching/detaching portion 152 and drives the bending wire 160.

As illustrated in FIG. 7, the wire driving unit 250 includes a vertical bending wire driving unit (first actuator) 251 and a horizontal bending wire driving unit (second actuator) 252.

The vertical bending wire driving unit 251 is a mechanism which is coupled with the vertical bending wire attaching/detaching portion 151 and drives the wires (the upper bending wire 161u and the lower bending wire 161d) that bend the bending portion 112 in the UD direction.

The horizontal bending wire driving unit 252 is a mechanism which is coupled with the horizontal bending wire attaching/detaching portion 152 and drives the wires (the left bending wire 1611 and the right bending wire 161r) that bend the bending portion 112 in the LR direction.

The horizontal bending wire driving unit 252 has the same structure as the vertical bending wire driving unit 251, and therefore will not be illustrated or described.

As illustrated in FIG. 8, the vertical bending wire driving unit 251 includes a support member (housing) 255, an upper bending wire driving unit 256, a lower bending wire driving unit 257, and an attachment/detachment sensor 259.

The upper bending wire driving unit 256 is coupled with the first rotation drum 156 of the vertical bending wire attaching/detaching portion 151 and drives the upper bending wire 161u. The upper bending wire driving unit 256 includes a first shaft 256a, a first coupled portion 256c, a first elastic member 256s, a first motor unit 256b, and a first torque sensor 256e.

The first shaft 256a is supported by the first motor unit 256b to be rotatable around the first shaft rotation axis 256r and advanceable and retractable in the longitudinal direction A. When the first detachable portion 1501 of the endoscope 100 is attached to the drive device 200, the first shaft rotation axis 256r coincides with the first drum rotation axis 156r.

The first coupled portion 256c is a disk member that rotates around the first shaft rotation axis 256r. The first coupled portion 256c is fixed to the distal end of the first shaft 256a and rotates integrally with the first shaft 256a. As illustrated in FIG. 8, the first coupled portion 256c is exposed to the distal end side of the vertical bending wire driving unit 251. Two first fitting concave portions 256d are formed on the distal end side surface of the first coupled portion 256c. Two first fitting concave portions 256d are formed on both sides with the first shaft rotation axis 256r interposed therebetween.

As illustrated in FIG. 9, the first fitting convex portion 156d and the first fitting concave portion 256d are fitted to each other, and the first coupling portion 156c and the first coupled portion 256c are coupled with each other. As a result, the rotation of the first shaft 256a by the first motor unit 256b is transmitted to the first rotation drum 156. When the first shaft 256a rotates clockwise as viewed from the distal end side to the proximal end side, the upper bending wire 161u is pulled. Conversely, when the first shaft 256a rotates counterclockwise, the upper bending wire 161u is delivered.

The first elastic member 256s is, for example, a compression spring, and has a distal end portion in contact with the first coupled portion 256c and a proximal end portion in contact with the support member 255. The first elastic member 256s urges the first coupled portion 256c toward the distal end side A1. As illustrated in FIG. 9, when the first coupling portion 156c is attached, the first coupled portion 256c moves to the proximal end side A2 together with the first shaft 256a.

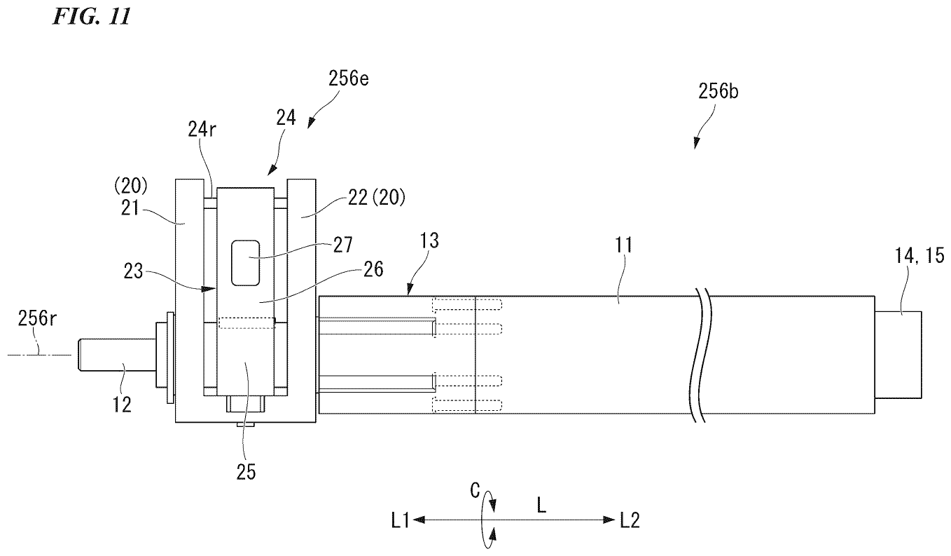

FIG. 11 is a diagram illustrating the first motor unit 256b and the first torque sensor 256e.

The first motor unit (motor device) 256b rotates the first shaft 256a around the first shaft rotation axis 256r. The first motor unit 256b includes a motor 11, a drive shaft 12, a support member 13, a motor driver 14, and a motor encoder 15.

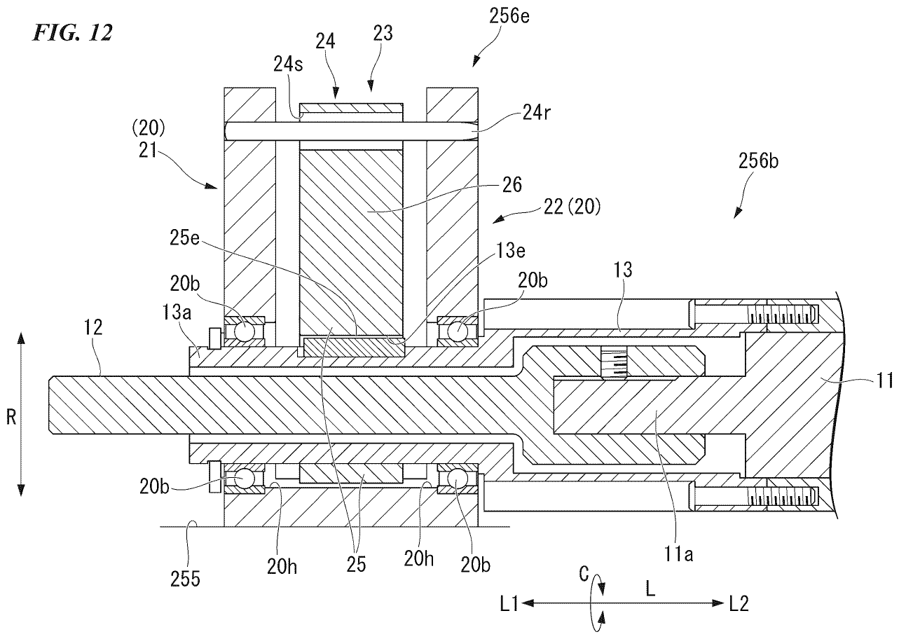

FIG. 12 is a cross-sectional view of the first motor unit 256b and the first torque sensor 256e.

The motor 11 is, for example, a servo motor and rotationally drives the connected drive shaft 12. The motor 11 includes a motor rotation shaft 11a that rotationally drives the drive shaft 12.

The drive shaft 12 is a shaft member extending in the axial direction L and is rotatable in the circumferential direction C. The drive shaft 12 is connected to the first shaft 256a and rotates the first shaft 256a around the first shaft rotation axis 256r. In the following description, in the axial direction L of the drive shaft 12, the side connected to the first shaft 256a is referred to as the “distal end side L1”, and the side opposite to the distal end side L1 is referred to as the “proximal end side L2”.

The support member 13 supports the motor 11. In the support member 13, a substantially cylindrical insertion portion 13a is provided at the distal end side L1. A convex or concave engagement portion 13e is formed on the outer periphery of the insertion portion 13a. The drive shaft 12 penetrates the insertion portion 13a of the support member 13. That is, the insertion portion 13a of the support member 13 is disposed around the drive shaft 12 along the circumferential direction C of the drive shaft 12.

In this embodiment, the support member 13 is fixed to the support member (housing) 255 via a fixed member 20 to be described later.

The motor driver 14 drives the motor 11. The motor driver 14 is controlled by the drive controller 260.

The motor encoder 15 detects the rotation angle of the drive shaft 12. The detected rotation angle of the drive shaft 12 is acquired by the drive controller 260.

The first torque sensor 256e detects the rotational torque about the drive shaft 12 and the first shaft 256a about the first shaft rotation axis 256r. The detection result of the first torque sensor 256e is acquired by the drive controller 260. The first torque sensor 256e includes the fixed member 20, a flexure body 23, and a strain sensor 27.

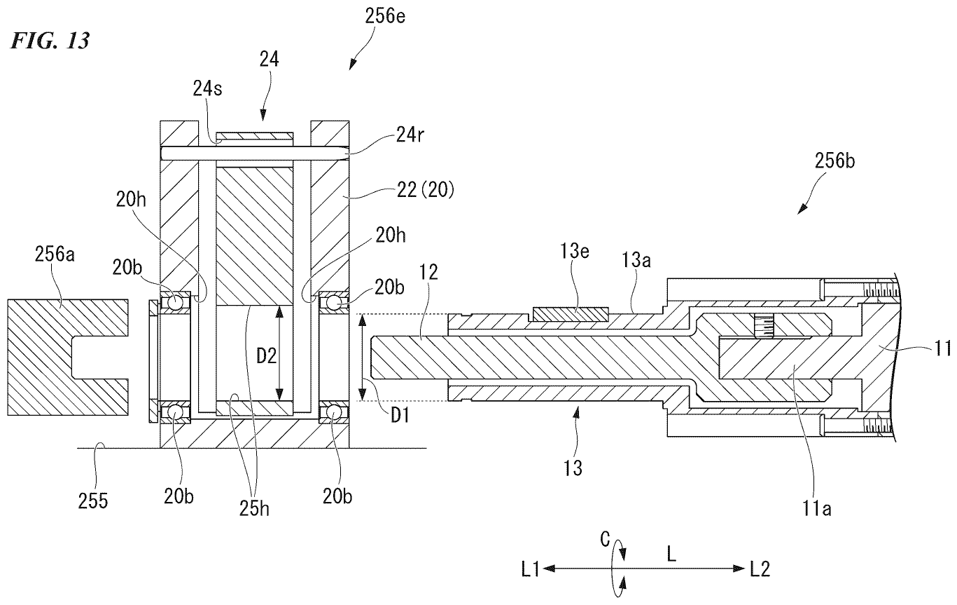

FIG. 13 is a cross-sectional view of the first motor unit 256b detached from the fixed member 20.

The fixed member 20 is a member fixed to the support member (housing) 255, and is connected to a first connection portion (connector) 24 to be described later. That is, the fixed member 20 fixes the first connection portion 24 to the support member (housing) 255.

In this embodiment, the fixed member 20 detachably supports the support member 13. Specifically, the fixed member 20 has a through hole 20h through which the support member 13 is insertable along the axial direction L. The through hole 20h has a bearing 20b which supports the attached support member 13 to be rotatable in the circumferential direction C.

The fixed member 20 has a first fixed member 21 and a second fixed member 22. The first fixed member 21 and the second fixed member 22 are members having the same shape. The first fixed member 21 is disposed closer to the distal end side L1 than the flexure body 23. The second fixed member 22 is disposed closer to the proximal end side L2 than the flexure body 23. The first fixed member 21 and the second fixed member 22 are arranged on both sides of the flexure body 23 in the axial direction L. The fixed member 20 may have any one of the first fixed member 21 and the second fixed member 22.

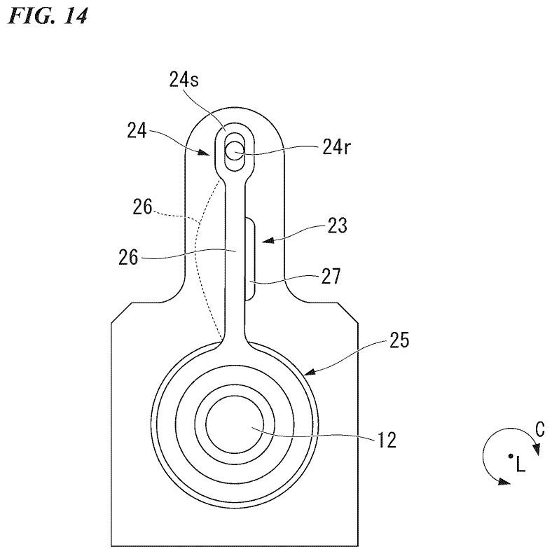

FIG. 14 is a diagram illustrating the flexure body 23.

The flexure body 23 has a first connection portion (connector) 24, a second connection portion (connector) 25, and a flexure portion 26. The flexure portion 26 is provided between the first connection portion 24 and the second connection portion 25. The flexure body 23 is formed as a member in which at least flexure portion 26 is elastically deformable.

In this embodiment, the flexure body 23 extends in the radial direction R with respect to the axial direction L of the drive shaft 12, and the first connection portion 24 and the second connection portion 25 are arranged along the radial direction R. The first connection portion 24 is disposed on the outside in the radial direction R, and the second connection portion 25 is disposed on the inside in the radial direction R.

The first connection portion 24 is formed at one end of the flexure body 23 in the longitudinal direction. The first connection portion 24 is connected to the fixed member 20a to be fixed to the support member (housing) 255.

In this embodiment, the first connection portion 24 has a connection rod 24r which is connected to the fixed member 20 and a connection support portion 24s which supports the connection rod 24r. The connection support portion 24s is formed in a ring shape through which the connection rod 24r is insertable.

The second connection portion 25 is formed at the other end of the flexure body 23 in the longitudinal direction. The second connection portion 25 is formed in a ring shape, and has an insertion hole 25h into which the insertion portion 13a of the support member 13 is insertable in the axial direction L.

The second connection portion 25 is detachably connected to the insertion portion 13a of the support member 13 by inserting the insertion portion 13a of the support member 13 into the insertion hole 25h. As illustrated in FIG. 13, the outer diameter D1 of the insertion portion 13a of the support member 13 is smaller than the inner diameter D2 of the insertion hole 25h.

In this embodiment, the insertion hole 25h penetrates the second connection portion 25 in the axial direction L. As illustrated in FIGS. 12 and 13, the through hole 20h and the insertion hole 25h are aligned along the axial direction L. Therefore, when the support member 13 is attached to the fixed member 20, the insertion portion 13a of the support member 13 is connected to the second connection portion 25, and the drive shaft 12 is connected to the first shaft 256a.

As illustrated in FIG. 12, when the insertion portion 13a of the support member 13 is attached to the second connection portion 25, the reaction force of the torque of the drive shaft 12 is transmitted to the second connection portion 25 via the support member 13.

As illustrated in FIG. 13, when the support member 13 is detached from the fixed member 20 and the second connection portion 25, the first motor unit 256b can be detached from the upper bending wire driving unit 256. When the motor 11 breaks down or the motor 11 wears out, only the first motor unit 256b can be replaced.

A convex or concave engagement portion 25e is formed on the inner periphery of the insertion hole 25h. When the insertion portion 13a is inserted into the insertion hole 25h, the engagement portion 25e of the insertion hole 25h and the engagement portion 13e of the insertion portion 13a engage with each other to fix the relative position between the second connection portion 25 and the insertion portion 13a in the circumferential direction C.

The flexure portion 26 is provided between the first connection portion 24 fixed to the support member (housing) 255 and the second connection portion 25 to which the reaction force of the torque of the drive shaft 12 is transmitted. Therefore, the flexure portion 26 is distorted in response to the reaction force of the torque of the drive shaft 12 as illustrated in FIG. 14.

The strain sensor 27 is provided in the flexure portion 26, and detects the strain of the flexure portion 26. The detected strain is acquired by the drive controller 260. The drive controller 260 can calculate the torque applied to the drive shaft 12 based on the acquired strain.

The lower bending wire driving unit 257 is coupled with the second rotation drum 157 of the vertical bending wire attaching/detaching portion 151 and drives the lower bending wire 161d. The lower bending wire driving unit 257 has a second shaft 257a, a second coupled portion 257c, a second elastic member 257s, a second motor unit 257b, and a second torque sensor 257e.

The second shaft 257a is supported by the second motor unit 257b to be rotatable around the second shaft rotation axis 257r and advanceable and retractable in the longitudinal direction A. When the first detachable portion 1501 of the endoscope 100 is attached to the drive device 200, the second shaft rotation axis 257r coincides with the second drum rotation axis 157r.

The second coupled portion 257c is a disk member that rotates around the second shaft rotation axis 257r. The second coupled portion 257c is fixed to the distal end of the second shaft 257a and rotates integrally with the second shaft 257a. As illustrated in FIG. 8, the second coupled portion 257c is exposed to the distal end side of the vertical bending wire driving unit 251. Two second fitting concave portions 257d are formed on the distal end side surface of the second coupled portion 257c. Two second fitting concave portions 257d are formed on both sides with the second shaft rotation axis 257r interposed therebetween.

As illustrated in FIG. 9, the second fitting convex portion 157d and the second fitting concave portion 257d are fitted to each other, and the second coupling portion 157c and the second coupled portion 257c are coupled with each other. As a result, the rotation of the second shaft 257a by the second motor unit 257b is transmitted to the second rotation drum 157. When the second shaft 257a rotates counterclockwise as viewed from the distal end side to the proximal end side, the lower bending wire 161d is pulled. Conversely, when the second shaft 257a rotates clockwise, the lower bending wire 161d is delivered.

The second elastic member 257s is, for example, a compression spring, and has a distal end portion in contact with the second coupled portion 257c and a proximal end portion in contact with the support member 255. The second elastic member 257s urges the second coupled portion 257c toward the distal end side A1. As illustrated in FIG. 9, when the second coupling portion 157c is attached, the second coupled portion 257c moves to the proximal end side A2 together with the second shaft 257a.

As illustrated in FIG. 9, the attachment/detachment sensor 259 detects the engagement and non-engagement with the attachment/detachment detection dog to detect whether the vertical bending wire attaching/detaching portion 151 is attached to and detached from the vertical bending wire driving unit 251. The detection result of the attachment/detachment sensor 259 is acquired by the drive controller 260.

The second motor unit (motor device) 257b rotates the second shaft 257a around the second shaft rotation axis 257r. The second motor unit 257b includes the motor 11, the drive shaft 12, the support member 13, the motor driver 14, and the motor encoder 15 as in the first motor unit 256b.

The second torque sensor 257e detects the rotational torque about the second shaft rotation axis 257r of the second shaft 257a. The detection result of the second torque sensor 257e is acquired by the drive controller 260. The second torque sensor 257e includes the fixed member 20, the flexure body 23, and the strain sensor 27 as in the first torque sensor 256e.

With the above structure, when the vertical bending wire attaching/detaching portion 151 is attached to the vertical bending wire driving unit 251, the upper bending wire driving unit 256 can independently drive the upper bending wire 161u, and the lower bending wire driving unit 257 can independently drive the lower bending wire 161d. Therefore, even if the distance from the bending portion 112 of the endoscope 100 to the drive device 200 is longer than that of a conventional flexible endoscope, the bending operation of the bending portion 112 can be controlled with high precision.

The drive controller 260 controls the entire drive device 200. The drive controller 260 acquires an operation input received by the operation receiving unit 220. The drive controller 260 controls the air supply/suction drive unit 230 and the wire driving unit 250 based on the acquired operation input.

The drive controller 260 is a computer including a processor 261, a memory 262, a storage unit 263 capable of storing a program and data, and an input/output control unit 264 and capable of executing a program. The functions of the drive controller 260 are realized by the processor executing a program. At least a part of the functions of the drive controller 260 may be realized by a dedicated logic circuit.

The drive controller 260 desirably has high calculation performance in order to control the plurality of motors that drive the plurality of bending wires 160 with high precision.

Furthermore, the drive controller 260 may further include a configuration other than the processor 261, the memory 262, the storage unit 263, and the input/output control unit 264. For example, the drive controller 260 may further include an image calculation unit that performs a part or all of the image processing and image recognition processing. By further including the image calculation unit, the drive controller 260 can perform specific image processing and image recognition processing at high speed. The image calculation unit may be mounted on a separate hardware device connected via a communication line.

### [Operation Device 300]

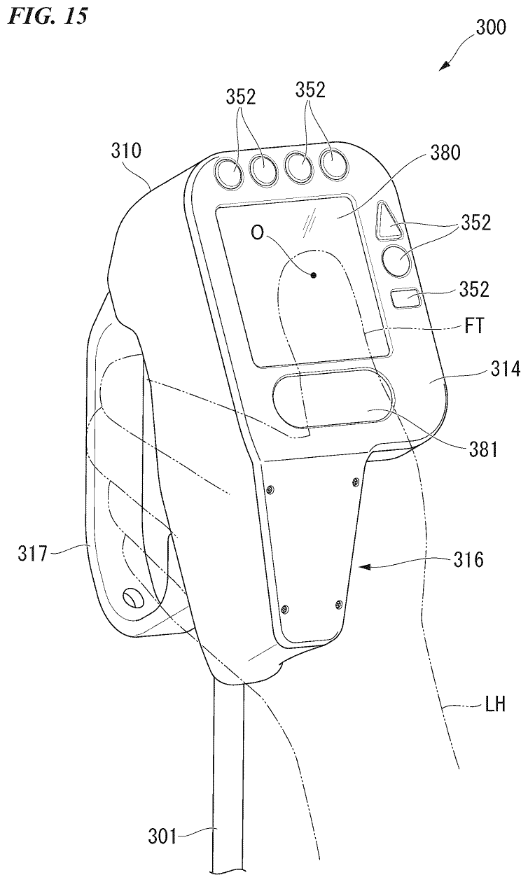

FIG. 15 is a perspective view of the operation device 300.

The operation device 300 is a device to which an operation for driving the endoscope 100 is input. The input operation input is transmitted to the drive device 200 via the operation cable 301. The operation device 300 may be capable of communicating with the drive device 200 via wireless communication instead of wired communication.

The operation device 300 includes an operation unit body 310, an air supply button, a suction button, various buttons 352, a touch pad 380, and a touch sensor 381.

The operation unit body 310 is formed in a substantially rod shape so that the surgeon S can hold the operation unit body in his/her left hand LH. The operation unit body 310 has a touch pad support portion 314 provided on the upper side, a grip portion 316 provided on the lower side, and a handle 317 provided on the rear side. As illustrated in FIG. 11, the surgeon S can operate the touch pad 380 with the thumb FT of the left hand LH while holding the grip portion 316 with the left hand LH.

The touch pad 380 is a touch-sensitive interface through which a bending operation or the like is input to the bending portion 112. The touch pad 380 may be a touch panel.

### [Video Control Device 500]

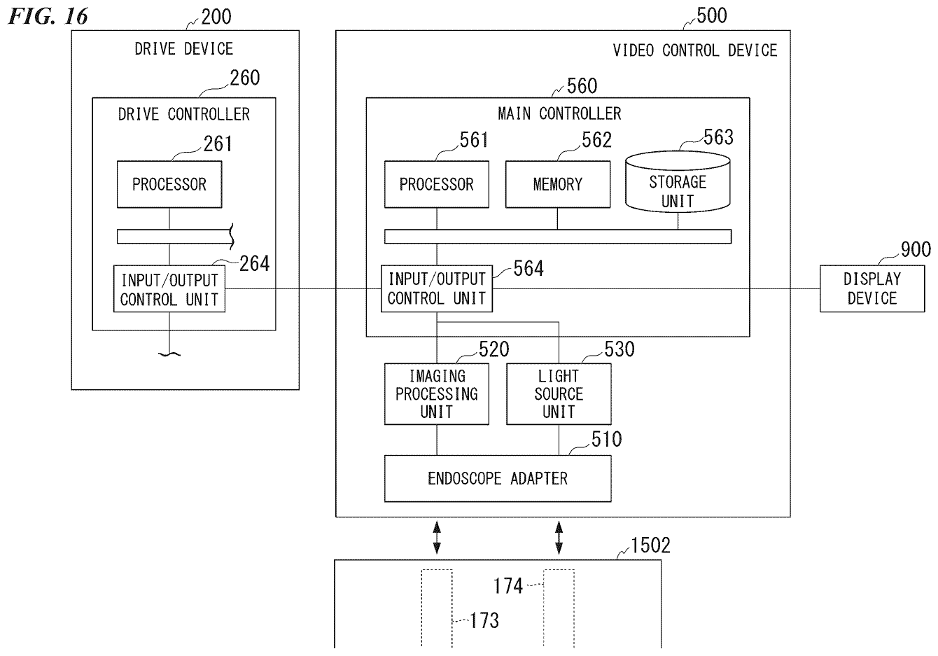

FIG. 16 is a functional block diagram of the video control device 500.

The video control device 500 controls the electric endoscope system 1000. The video control device 500 includes a third adapter 510, an imaging processing unit 520, a light source unit 530, and a main controller 560.

The third adapter 510 is an adapter to which the second detachable portion 1502 of the endoscope 100 is detachably connected.

The imaging processing unit 520 converts an imaging signal acquired from the imaging unit 111c of the distal end portion 111 via the imaging cable 173 into a captured image.

The light source unit 530 generates illumination light to be irradiated onto the imaging target. The illumination light generated by the light source unit 530 is guided to the illumination unit 111b of the distal end portion 111 via the light guide 174.

The main controller 560 is a computer including a processor 561, a memory 562, a storage unit 563 capable of storing a program and data, and an input/output control unit 564 and capable of executing a program. The functions of the main controller 560 are realized by the processor 561 executing a program. At least a part of the functions of the main controller 560 may be realized by a dedicated logic circuit.

The main controller 560 includes the processor 561, the memory 562 capable of reading a program, the storage unit 563, and the input/output control unit 564.

The storage unit 563 is a non-volatile recording medium that stores the above programs and necessary data. The storage unit 563 is configured, for example, by a ROM, a hard disk, and the like. The program recorded in the storage unit 563 is read into the memory 562 and executed by the processor 561.

The input/output control unit 564 is connected to the imaging processing unit 520, the light source unit 530, the drive device 200, the display device 900, an input device (not illustrated), and a network device (not illustrated). The input/output control unit 564 transmits and receives data and control signals to and from connected devices based on the control of the processor 561.

The main controller 560 can perform image processing on the captured image acquired by the imaging processing unit 520. The main controller 560 can generate GUI images and CG images for the purpose of providing information to the surgeon S. The main controller 560 can display the captured images, GUI images, and CG images on the display device 900.

The main controller 560 is not limited to an integrated hardware device. For example, the main controller 560 may be configured by separating a part of the main controller into separate hardware devices and connecting the separated hardware devices via a communication line. For example, the main controller 560 may be a cloud system in which the separated storage units 563 are connected via a communication line.

The main controller 560 may further have a configuration other than the processor 561, the memory 562, the storage unit 563, and the input/output control unit 564 illustrated in FIG. 12. For example, the main controller 560 may further include an image calculation unit that performs a part or all of the image processing and image recognition processing that were previously performed by the processor 561. By further including the image calculation unit, the main controller 560 can perform specific image processing and image recognition processing at high speed. The image calculation unit may be mounted on a separate hardware device connected via a communication line.

### [Operation of Electric Endoscope System 1000]

Next, the operation of the electric endoscope system 1000 of this embodiment will be described. Specifically, a procedure for observing and treating an affected area formed on a lumen wall inside the large intestine using the electric endoscope system 1000 will be described.

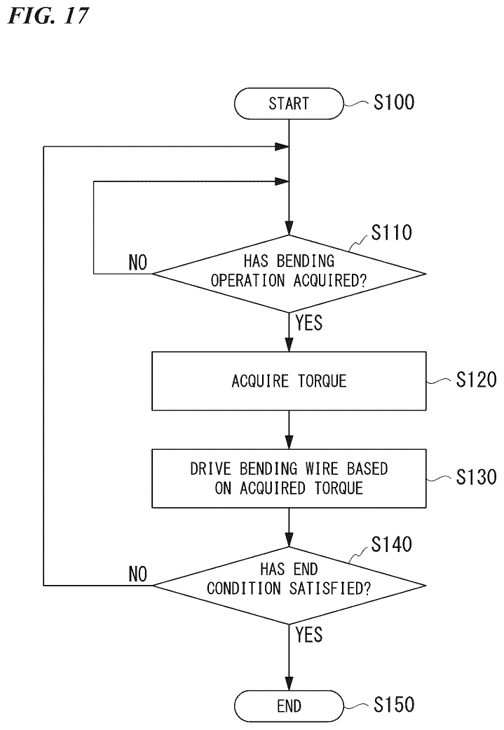

The following description will be given with reference to the control flowchart of the drive controller 260 of the control device 600 illustrated in FIG. 17. When the control device 600 is started, the drive controller 260 performs initialization and then starts bending drive control of the bending portion 112 (step S100). Next, the drive controller 260 (mainly the processor 261) performs step S110.

The surgeon S inserts the insertion portion 110 of the endoscope 100 into the large intestine of the patient P through the anus. While observing the captured image displayed on the display device 900, the surgeon S operates the internal flexible portion 119 with his/her right hand RH, and moves the insertion portion 110 to bring the distal end portion 111 closer to the affected area. Further, the surgeon S operates the operation device 300 with the left hand LH to input a bending operation for the bending portion 112.

### <Step S110>

In step S110, the drive controller 260 acquires a bending operation for the bending portion 112 from the operation device 300.

### <Step S120>

In step S120, the drive controller 260 calculates the torque of the drive shaft 12 from the detection results of the strain sensor 27 and the torque sensors provided in the wire driving unit 250, such as the first torque sensor 256e and the second torque sensor 257e.

### <Step S130>

In step S130, the drive controller 260 drives the bending wire 160 based on the acquired torque in order to realize the received bending operation.

### <Step S140>

In step S140, the drive controller 260 performs an end determination of the bending drive control. When the drive controller 260 determines not to end the bending drive control, the drive controller performs step S110. When the drive controller 260 determines to end the bending drive control, the drive controller performs step S140 to end the bending drive control.

The above bending drive control is performed on each of four bending wires 160 (the upper bending wire 161u, the lower bending wire 161d, the left bending wire 1611, and the right bending wire 161r).

Furthermore, the above bending drive control may be performed when the main controller 560 (mainly the processor 561) controls the wire driving unit (actuator) 250.

According to the electric endoscope system 1000 of this embodiment, the torque of the motor 11 driving the endoscope 100 can be sensed, and the motor 11 can be easily replaced. The first motor unit 256b does not include a torque sensor directly connected to the drive shaft 12, but can indirectly acquire the torque of the drive shaft 12 by the first torque sensor 256e attached to the support member 13 supporting the motor 11. Further, since the first motor unit 256b can be attached to and detached from the first torque sensor 256e by a simple operation, it is possible to easily replace only the first motor unit 256b while leaving the first torque sensor 256e when the motor 11 breaks down or the motor 11 wears out. Other motor units such as the second motor unit 257b can also be easily replaced in the same manner.

Although the first embodiment of the present disclosure has been described in detail above with reference to the drawings, the specific configuration is not limited to this embodiment, and design changes and the like within the scope of the gist of the present disclosure are also included. Furthermore, the components illustrated in the above-described embodiment and modified examples can be appropriately combined to form a configuration.

### Modified Example 1

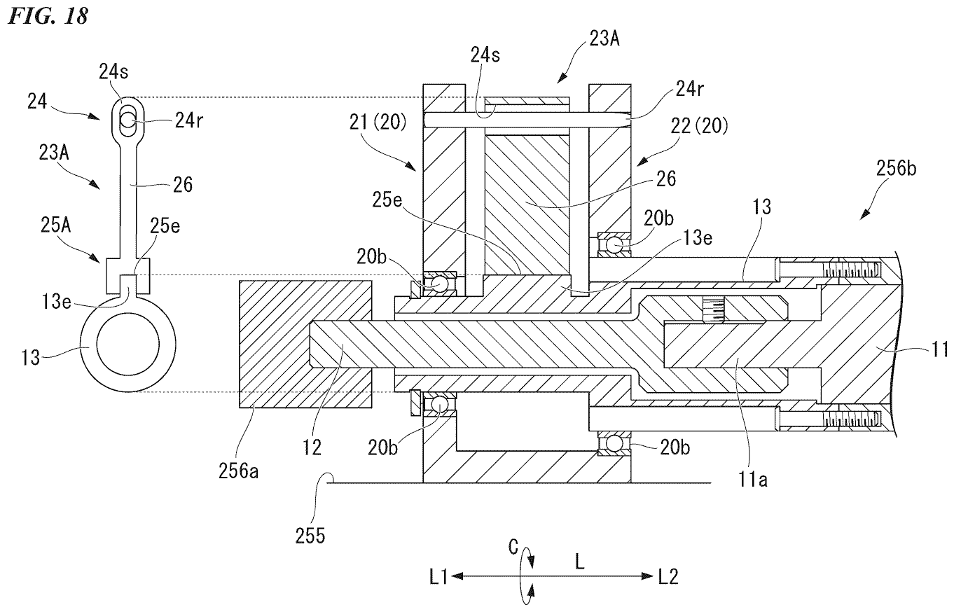

FIG. 18 is a diagram illustrating a flexure body 23A which is a modified example of the flexure body 23.

The flexure body 23A has the first connection portion 24, a second connection portion (connector) 25A, and the flexure portion 26. The second connection portion 25A is a modified example of the second connection portion 25. The second connection portion 25A is not formed in a ring shape and does not have the insertion hole 25h. The second connection portion 25A has an engagement portion 25e which engages with the engagement portion 13e of the insertion portion 13a. The second connection portion 25A is detachably connected to the insertion portion 13a of the support member 13 by engaging the engagement portion 13e of the insertion portion 13a with the engagement portion 25e.

### Modified Example 2

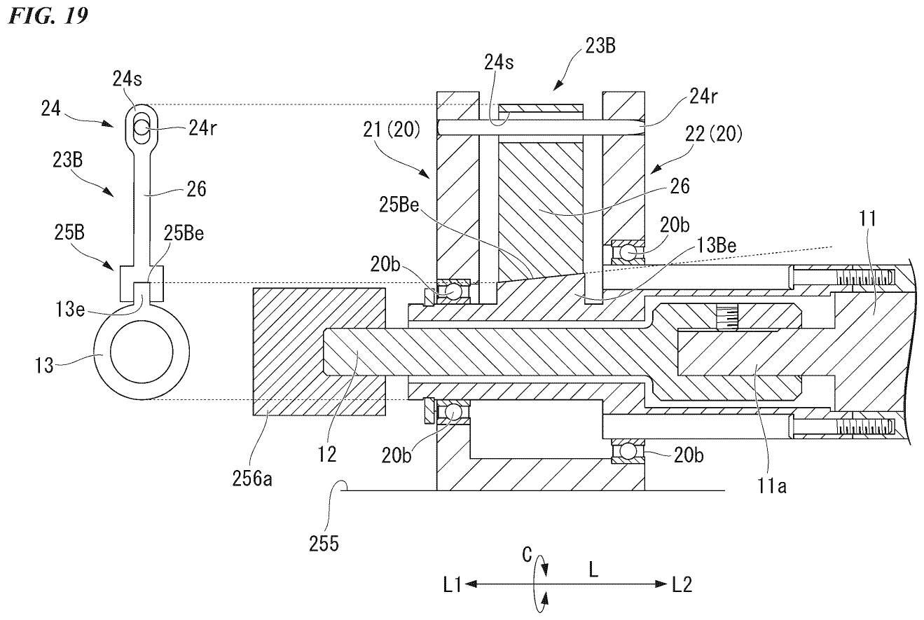

FIG. 19 is a diagram illustrating a flexure body 23B which is a modified example of the flexure body 23.

The flexure body 23B has the first connection portion 24, a second connection portion (connector) 25B, and the flexure portion 26. The second connection portion 25B is a modified example of the second connection portion 25A. The second connection portion 25B has an engagement portion 25Be which is a modified example of the engagement portion 25e. The engagement portion 25Be is formed in a convex shape or concave shape and is inclined with respect to the axial direction L. An engagement portion 13Be which is a modified example of the engagement portion 13e is formed in a convex shape or concave shape and is inclined with respect to the axial direction L. The engagement portion 25Be and the engagement portion 13Be are inclined to move away from each other outward in the radial direction R with respect to the rotation axis of the drive shaft 12 as they move from the distal end side L1 toward the proximal end side L2. Thus, the engagement portion 13Be and the engagement portion 25Be can be easily connected to each other from the proximal end side L2 in the axial direction L, and can be easily detached from each other. Accordingly, the motor unit including the first motor unit 256b or the second motor unit 257b is more easily attached and detached.

### Modified Example 3

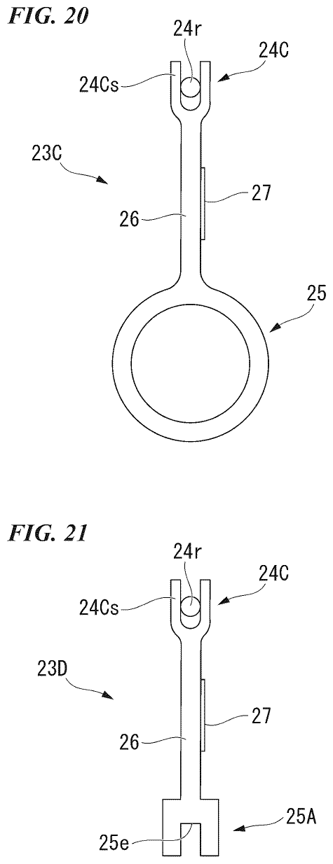

FIG. 20 is a diagram illustrating a flexure body 23C which is a modified example of the flexure body 23.

The flexure body 23C has a first connection portion (connector) 24C, the second connection portion 25, and the flexure portion 26. The first connection portion 24C is a modified example of the first connection portion 24. The first connection portion 24C has a connection rod 24r which is connected to the fixed member 20 and a connection support portion 24Cs which supports the connection rod 24r. The connection support portion 24Cs is formed in a U shape through which the connection rod 24r is insertable. The connection support portion 24Cs can easily connect the connection rod 24r compared to the ring-shaped connection support portion 24s.

### Modified Example 4

FIG. 21 is a diagram illustrating a flexure body 23D which is a modified example of the flexure body 23.

The flexure body 23D has the first connection portion 24C, the second connection portion 25A, and the flexure portion 26. The flexure body 23D can be easily detached from the fixed member 20 and replaced, and is therefore highly maintainable.

### Second Embodiment

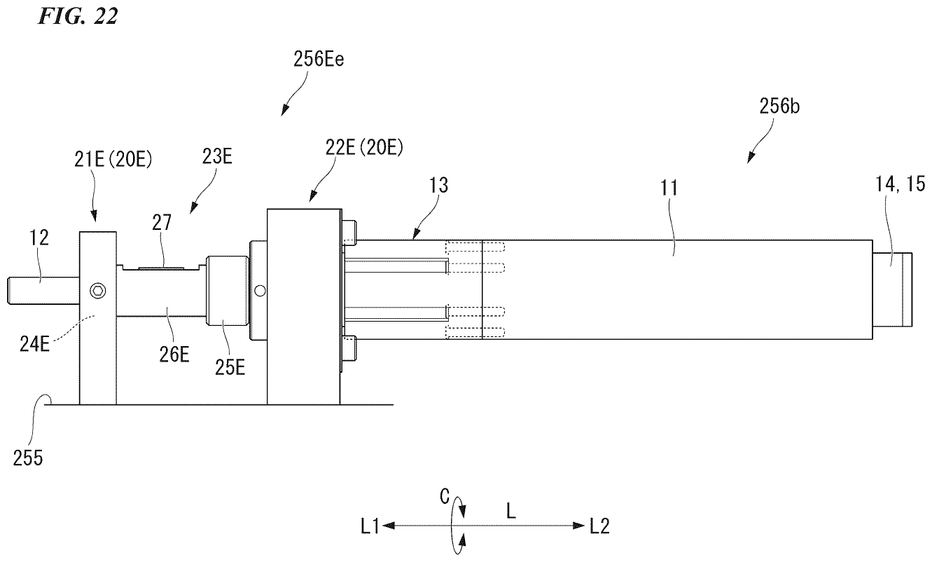

An electric endoscope system 1000E according to a second embodiment of the present disclosure will be described with reference to FIGS. 22 to 25. In the following description, the same components as those already described will be denoted by the same reference numerals and the description thereof will be omitted.

### [Electric Endoscope System 1000E]

As illustrated in FIG. 1, the electric endoscope system 1000E includes the endoscope 100, a drive device 200E, the operation device 300, the treatment tool 400, the video control device 500, and the display device 900. The drive device 200E and the video control device 500 constitute a control device 600E that controls the electric endoscope system 1000E.

### [Drive Device 200E]

The drive device 200E includes the adapter 210, the operation receiving unit 220, the air supply/suction drive unit 230, a wire driving unit (actuator) 250E, and the drive controller 260.

The wire driving unit (actuator) 250E includes a different torque sensor compared to the wire driving unit 250 of the first embodiment. As illustrated in FIG. 7, the wire driving unit 250E includes the vertical bending wire driving unit (first actuator) 251E and a horizontal bending wire driving unit (second actuator) 252E.

The horizontal bending wire driving unit 252E has the same structure as the vertical bending wire driving unit 251E, and therefore will not be illustrated or described.

The vertical bending wire driving unit 251E includes the support member (housing) 255, an upper bending wire driving unit 256E, a lower bending wire driving unit 257E, and the attachment/detachment sensor 259.

The upper bending wire driving unit 256E is coupled with the first rotation drum 156 of the vertical bending wire attaching/detaching portion 151 and drives the upper bending wire 161u. The upper bending wire driving unit 256E includes the first shaft 256a, the first coupled portion 256c, the first elastic member 256s, the first motor unit 256b, and a first torque sensor 256Ee.

The lower bending wire driving unit 257E has the same structure as the upper bending wire driving unit 256E, and therefore will not be illustrated or described.

FIG. 22 is a diagram illustrating the first motor unit 256b and the first torque sensor 256Ee.

In this embodiment, the support member 13 is fixed to the support member (housing) 255 via a second fixed member 22E to be described later.

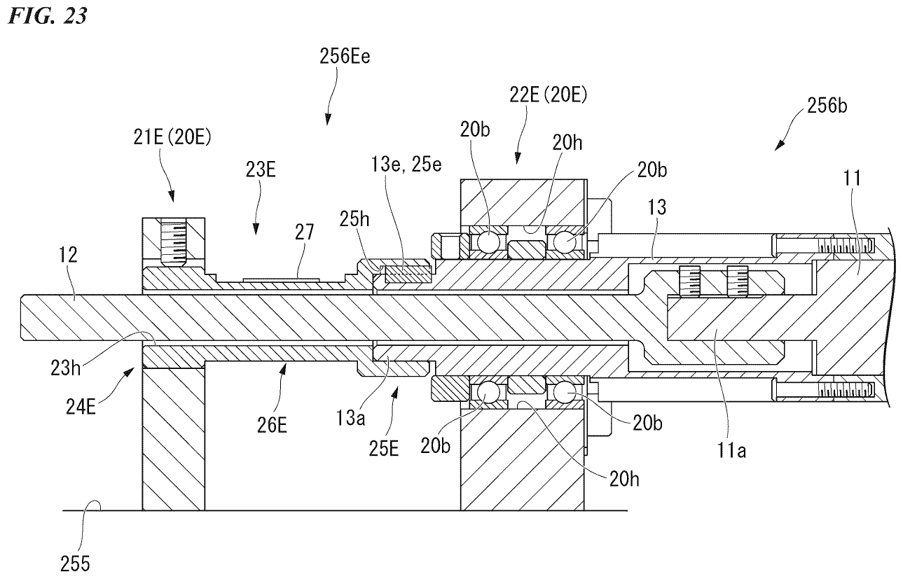

FIG. 23 is a cross-sectional view of the first motor unit 256b and the first torque sensor 256Ee.

The first torque sensor 256Ee detects the rotational torque of the drive shaft 12 and the first shaft 256a about the first shaft rotation axis 256r. The detection result of the first torque sensor 256Ee is acquired by the drive controller 260. The first torque sensor 256Ee includes a fixed member 20E, a flexure body 23E, and the strain sensor 27.

The fixed member 20E is a member fixed to the support member (housing) 255 and has a first fixed member 21E and a second fixed member 22E.

In this embodiment, the first fixed member 21E is connected to a first connection portion (connector) 24E to be described later. That is, the first fixed member 21E fixes the first connection portion 24E to the support member (housing) 255.

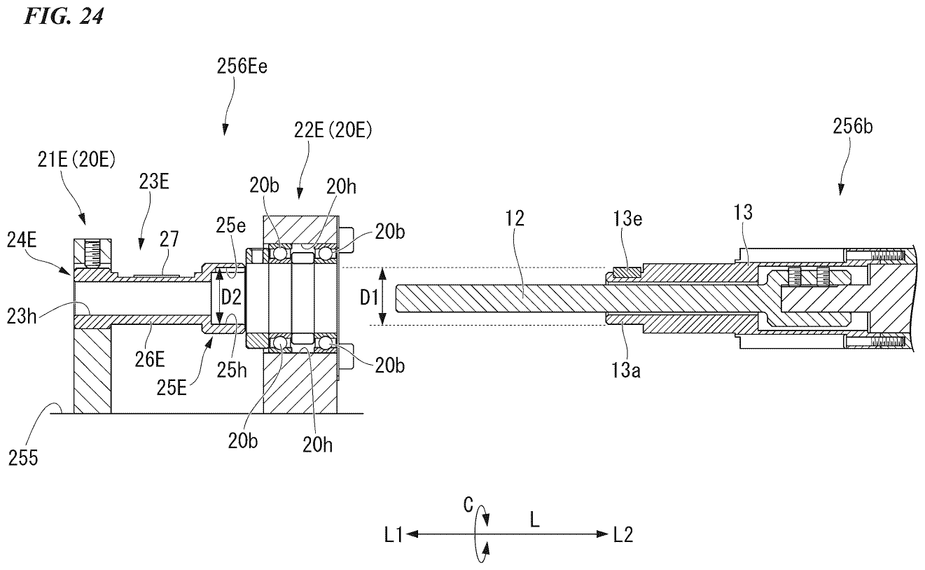

FIG. 24 is a cross-sectional view of the first motor unit 256b detached from the fixed member 20E.

In this embodiment, the second fixed member 22E detachably supports the support member 13. Specifically, the second fixed member 22E has the through hole 20h through which the support member 13 is insertable along the axial direction L. The through hole 20h has the bearing 20b which supports the attached support member 13 to be rotatable in the circumferential direction C.

Furthermore, when other members can sufficiently support the support member 13, the second fixed member 22E is not necessary.

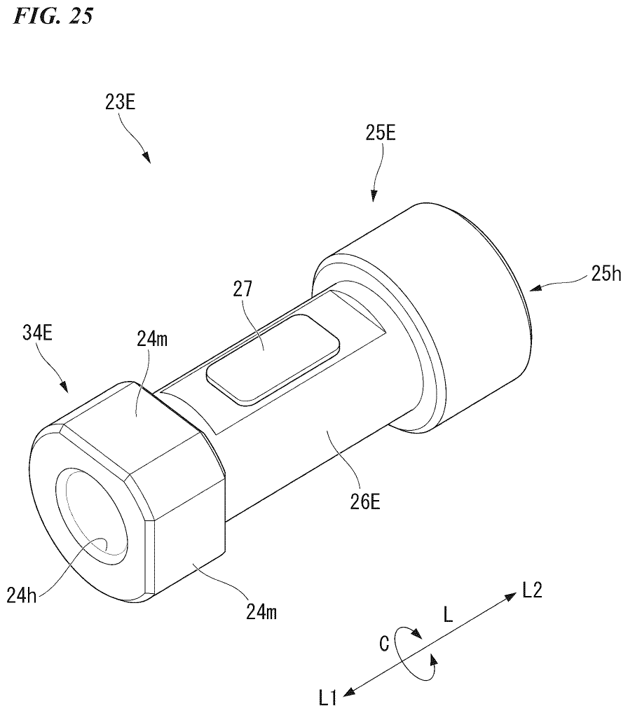

FIG. 25 is a perspective view of the flexure body 23E.

The flexure body 23E has a first connection portion (connector) 24E, a second connection portion (connector) 25E, and a flexure portion 26E. The flexure portion 26E is provided between the first connection portion 24E and the second connection portion 25E. The flexure body 23E is formed as a member in which at least the flexure portion 26E is elastically deformable.

In this embodiment, the flexure body 23E extends in the axial direction L of the drive shaft 12. The first connection portion 24E and the second connection portion 25E are arranged along the axial direction L. The first connection portion 24E is disposed on the distal end side L1 in the axial direction L, and the second connection portion 25E is disposed on the proximal end side L2 in the axial direction L. The flexure body 23E has a through hole 23h that penetrates in the axial direction L. The drive shaft 12 is insertable through the through hole 23h.

The first connection portion 24E is formed at one end of the flexure body 23E in the longitudinal direction. The first connection portion 24E is connected to the first fixed member 21E to be fixed to the support member (housing) 255.

In this embodiment, the first connection portion 24E has a notch portion 24n on the outer peripheral surface, and is connected to the first fixed member 21E not to be rotatable in the circumferential direction C.

The second connection portion 25E is formed at the other end of the flexure body 23E in the longitudinal direction. The second connection portion 25E is formed in a ring shape and has the insertion hole 25h through which the insertion portion 13a of the support member 13 is insertable along the axial direction L.

The second connection portion 25E is detachably connected to the insertion portion 13a of the support member 13 by inserting the insertion portion 13a of the support member 13 into the insertion hole 25h. As illustrated in FIG. 24, the outer diameter D1 of the insertion portion 13a of the support member 13 is smaller than the inner diameter D2 of the insertion hole 25h.

In this embodiment, the insertion hole 25h communicates with the through hole 23h penetrating the flexure body 23E in the axial direction L. As illustrated in FIGS. 23 and 24, the through hole 20h and the insertion hole 25h are arranged along the axial direction L. Therefore, when the support member 13 is attached to the second fixed member 22E, the insertion portion 13a of the support member 13 is connected to the second connection portion 25E, and the drive shaft 12 penetrates the through hole 23h to be connected to the first shaft 256a. At this time, the drive shaft 12 penetrates the first connection portion 24E, the second connection portion 25E, and the flexure body 23E.

As illustrated in FIG. 23, when the insertion portion 13a of the support member 13 is attached to the second connection portion 25E, the reaction force of the torque of the drive shaft 12 is transmitted to the second connection portion 25E via the support member 13.

As illustrated in FIG. 24, when the support member 13 is detached from the second fixed member 22E and the second connection portion 25E, the first motor unit 256b can be detached from the upper bending wire driving unit 256. When the motor 11 breaks down or the motor 11 wears out, only the first motor unit 256b can be replaced.

The convex or concave engagement portion 25e is formed on the inner periphery of the insertion hole 25h. When the insertion portion 13a is inserted into the insertion hole 25h, the engagement portion 25e of the insertion hole 25h and the engagement portion 13e of the insertion portion 13a engage with each other to fix the relative position between the second connection portion 25E and the insertion portion 13a in the circumferential direction C.

The flexure portion 26E is provided between the first connection portion 24E fixed to the support member (housing) 255 and the second connection portion 25E to which the reaction force of the torque of the drive shaft 12 is transmitted. Therefore, the flexure portion 26E is twisted and distorted in response to the reaction force of the torque of the drive shaft 12.

The strain sensor 27 is provided in the flexure portion 26E, and detects the strain of the flexure portion 26E. The detected strain is acquired by the drive controller 260. The drive controller 260 can calculate the torque applied to the drive shaft 12 based on the acquired strain.

According to the electric endoscope system 1000E of this embodiment, the torque of the motor 11 driving the endoscope 100 can be sensed, and the motor 11 can be easily replaced. The first motor unit 256b does not include a torque sensor directly connected to the drive shaft 12, but can indirectly acquire the torque of the drive shaft 12 by the first torque sensor 256Ee attached to the support member 13 supporting the motor 11. Further, since the first motor unit 256b can be attached to and detached from the first torque sensor 256Ee by a simple operation, it is possible to easily replace only the first motor unit 256b while leaving the first torque sensor 256Ee when the motor 11 breaks down or the motor 11 wears out. Other motor units such as the second motor unit 257b can also be easily replaced in the same manner.

Although the second embodiment of the present disclosure has been described above in detail with reference to the drawings, the specific configuration is not limited to this embodiment, and design changes and the like within the scope of the present disclosure are also included. Furthermore, the components illustrated in the above-described embodiment and modified examples can be appropriately combined to form a configuration.

### MODIFIED EXAMPLES

In the above-described embodiments, for example, the upper bending wire 161u and the lower bending wire 161d are independently driven by different motor units (the first motor unit 256b and the second motor unit 257b). However, the upper bending wire 161u and the lower bending wire 161d may be driven in conjunction with each other by the same motor unit. The same applies to the left bending wire 1611 and the right bending wire 161r.

The programs in each embodiment may be recorded on a computer-readable recording medium, and the programs recorded on the recording medium may be read into a computer system and executed to realize the present disclosure. Furthermore, the “computer system” includes the OS and hardware such as peripheral devices. Further, the “computer-readable recording medium” refers to portable media such as flexible disks, optical magnetic disks, ROMs, and CD-ROMs, as well as storage devices such as hard disks built into computer systems. Moreover, the “computer-readable recording medium” may include a medium that dynamically holds a program for a short period of time, such as a communication line when transmitting a program via a network such as the Internet or a communication line such as a telephone line, and a medium that holds a program for a certain period of time, such as a volatile memory inside a computer system that is a server or client in such a case. Furthermore, the above program may be for realizing a part of the functions described above, and may further be capable of realizing the functions described above in combination with a program already recorded in the computer system.

The present disclosure can be applied to the drive device for the medical manipulator.

### Example 1

A drive device for a medical manipulator having a wire driving a movable portion, comprising:
a housing;
a motor which has a drive shaft rotating in a circumferential direction and drives the wire;
a support member that supports the motor;
a flexure body which has a first connection portion fixed to the housing, a second connection portion detachably connected to the support member, and a flexure portion provided between the first connection portion and the second connection portion; and
a strain sensor which is disposed in the flexure portion,
wherein the second connection portion fixes a relative position with respect to the support member in the circumferential direction when the support member is attached.

### Example 2

The drive device for the medical manipulator according to Example 1,
wherein the second connection portion has an insertion hole into which an insertion portion serving as at least a part of the support member is insertable, and
wherein an outer diameter of the insertion portion of the support member is smaller than an inner diameter of the insertion hole.

### Example 3

The drive device for the medical manipulator according to Example 2,
wherein the drive shaft penetrates the insertion portion of the support member.

### Example 4

The drive device for the medical manipulator according to Example 2,
wherein the insertion hole of the second connection portion has an engagement portion which fixes a relative position with respect to the insertion portion in the circumferential direction when the insertion portion is inserted into the insertion hole.

### Example 5

The drive device for the medical manipulator according to Example 2,
wherein the support member has an engagement portion which fixes a relative position with respect to the second connection portion in the circumferential direction when the support member is inserted into the insertion hole of the second connection portion.

### Example 6

The drive device for the medical manipulator according to Example 2, further comprising:
a fixed member that is fixed to the housing,
wherein the fixed member is connected to the first connection portion, and
wherein the fixed member has a through hole through which the support member is inserted and supports the support member to be detachable and rotatable in the circumferential direction.

### Example 7

The drive device for the medical manipulator according to Example 6,
wherein the insertion hole and the through hole are arranged along an axial direction of the drive shaft.

### Example 8

The drive device for the medical manipulator according to Example 1, further comprising:
a fixed member that is fixed to the housing,
wherein the fixed member is connected to the first connection portion,
wherein the fixed member supports the support member to be detachable and rotatable in the circumferential direction, and
wherein the first connection portion and the second connection portion of the flexure body are arranged along a radial direction of the drive shaft of the motor.

### Example 9

The drive device for the medical manipulator according to Example 8,
wherein the first connection portion is disposed on the outside in the radial direction, and
wherein the second connection portion is disposed on the inside in the radial direction.

### Example 10

The drive device for the medical manipulator according to Example 8,
wherein the second connection portion has an insertion hole into which an insertion portion serving as at least a part of the support member is insertable, and
wherein the second connection portion is formed in a ring shape into which the insertion portion of the support member is insertable.

### Example 11

The drive device for the medical manipulator according to Example 8, further comprising:
a first fixed member and a second fixed member that serve as the fixed member,
wherein the first fixed member and the second fixed member are arranged on both sides of the flexure body in an axial direction of the drive shaft.

### Example 12

The drive device for the medical manipulator according to Example 1, further comprising:
a first fixed member and a second fixed member that are fixed to the housing,
wherein the first fixed member is connected to the first connection portion,
wherein the second fixed member supports the support member to be detachable and rotatable in the circumferential direction, and
wherein the first connection portion and the second connection portion of the flexure body are arranged along an axial direction of the drive shaft.

### Example 13

The drive device for the medical manipulator according to Example 12,
wherein the first connection portion is disposed on a distal end side in the axial direction, and
wherein the second connection portion is disposed on a proximal end side in the axial direction.

### Example 14

The drive device for the medical manipulator according to Example 12,
wherein the drive shaft penetrates the first connection portion and the second connection portion.

### Example 15

A medical manipulator system comprising:
the drive device according to Example 1; and
the medical manipulator.

### Example 16

A motor device attached to a drive device driving a medical manipulator having a wire driving a movable portion, comprising:
a motor which has a drive shaft rotating in a circumferential direction and drives the wire; and
a support member that supports the motor,
wherein the support member is supported by the drive device to be detachable and rotatable in the circumferential direction, and
wherein the support member has an insertion portion which engages with a torque sensor included in the drive device when the support member is attached to the drive device, and fixes a relative position between the torque sensor and the support member in the circumferential direction.

### Example 17

The motor device for the medical manipulator according to Example 16,
wherein the drive shaft penetrates the insertion portion of the support member.

### Example 18

The motor device for the medical manipulator according to Example 16,
wherein the support member has an engagement portion which fixes a relative position with respect to the torque sensor in the circumferential direction when engaging with the torque sensor.

What is claimed is:

1. A drive device for a medical manipulator, comprising:
a housing;
a motor;
a support member that supports the motor;
a flexure body having a first connector fixed to the housing, a second connector detachably connected to the support member, and a flexure portion provided between the first connector and the second connector; and
a strain sensor disposed in the flexure portion,
wherein the second connector is fixed relative to the support member in a circumferential direction of the motor when the support member is attached.

2. The drive device for the medical manipulator according to claim 1,
wherein the second connector has an insertion hole into which an insertion portion serving as at least a part of the support member is insertable.

3. The drive device for the medical manipulator according to claim 2,
wherein a drive shaft of the motor penetrates the insertion portion of the support member.

4. The drive device for the medical manipulator according to claim 2,
wherein the insertion hole of the second connector has an engagement portion which is fixed relative to the insertion portion in the circumferential direction when the insertion portion is inserted into the insertion hole.

5. The drive device for the medical manipulator according to claim 2,
wherein the support member has an engagement portion which is fixed relative to the second connector in the circumferential direction when the insertion portion is inserted into the insertion hole.

6. The drive device for the medical manipulator according to claim 2, further comprising:
a fixed member that is fixed to the housing,
wherein the fixed member is connected to the first connector, and
wherein the fixed member has a through hole through which the support member is inserted and supports the support member to be detachable and rotatable in the circumferential direction.

7. The drive device for the medical manipulator according to claim 6,
wherein the insertion hole and the through hole are arranged along an axial direction of a drive shaft of the motor.

8. The drive device for the medical manipulator according to claim 1, further comprising:
a fixed member that is fixed to the housing,
wherein the fixed member is connected to the first connector,
wherein the fixed member supports the support member to be detachable and rotatable in the circumferential direction, and
wherein the first connector and the second connector of the flexure body are arranged along a radial direction of a drive shaft of the motor.

9. The drive device for the medical manipulator according to claim 8,
wherein the first connector is disposed on an outside in the radial direction, and
wherein the second connector is disposed on an inside in the radial direction.

10. The drive device for the medical manipulator according to claim 8,
wherein the second connector has an insertion hole into which an insertion portion serving as at least a part of the support member is insertable, and
wherein the second connector is formed in a ring shape into which the insertion portion of the support member is insertable.

11. The drive device for the medical manipulator according to claim 8, further comprising:
a first fixed member and a second fixed member that serve as the fixed member,
wherein the first fixed member and the second fixed member are arranged on both sides of the flexure body in an axial direction of the drive shaft.

12. The drive device for the medical manipulator according to claim 1, further comprising:
a first fixed member and a second fixed member that are fixed to the housing,
wherein the first fixed member is connected to the first connector,
wherein the second fixed member supports the support member to be detachable and rotatable in the circumferential direction, and
wherein the first connector and the second connector of the flexure body are arranged along an axial direction of a drive shaft of the motor.

13. The drive device for the medical manipulator according to claim 12,
wherein the first connector is disposed on a distal end side in the axial direction, and
wherein the second connector is disposed on a proximal end side in the axial direction.

14. The drive device for the medical manipulator according to claim 12,
wherein the drive shaft penetrates the first connector and the second connector.

15. A medical manipulator system comprising:
the drive device according to claim 1; and
the medical manipulator.

16. A motor device attached to a drive device for a medical manipulator, comprising:
a motor; and
a support member that supports the motor,
wherein the support member is supported by the drive device to be detachable and rotatable in a circumferential direction of the motor, and
the support member has an insertion portion which engages with a torque sensor included in the drive device when the support member is attached to the drive device, and the torque sensor is fixed relative to the support member in the circumferential direction.

17. The motor device for the medical manipulator according to claim 16,
wherein the drive shaft penetrates the insertion portion of the support member.

18. The motor device for the medical manipulator according to claim 16,
wherein the support member has an engagement portion which is fixed relative to the torque sensor in the circumferential direction when engaging with the torque sensor.

19. The drive device for the medical manipulator according to claim 2,
wherein the support member has a protrusion extending in a radial direction of the support member, the support member fixed relative to the second connector in the circumferential direction when the insertion portion is inserted into the insertion hole.

20. The drive device for the medical manipulator according to claim 19,
wherein the second connector has a recess contacting the protrusion to fix the support member relative to the second connector in the circumferential direction when the insertion portion is inserted into the insertion hole.
